(19) United States
(12) Patent Application Publication
(10) Pub. No.: US 2016/0198781 A1
(43) Pub. Date: Jul. 14, 2016

(54) Garment With Sound Absorbing Layer

(71) Applicants: Tara A. Israel, East Hampton, NY (US); Bonnie S. Schnitta, 29 Gann Road, NY (US)

(72) Inventors: Tara A. Israel, East Hampton, NY (US); Bonnie S. Schnitta, 29 Gann Road, NY (US)

(21) Appl. No.: 14/996,213

(22) Filed: Jan. 14, 2016

Related U.S. Application Data
(60) Provisional application No. 62/103,356, filed on Jan. 14, 2015.

(51) Int. Cl. A41D 31/00; A61F 11/14; A41D 23/00; A41D 1/04

(52) U.S. Cl. CPC A41D 31/0011; A41D 1/04; A61F 11/14; A41D 23/00; A41D 2200/20

(57) ABSTRACT

A garment engaged in a sound-reducing state includes a first garment portion formed with a sound-absorbing material and configured to surround a wearer's head and ears to form a substantially enclosed reduced-sound zone, when the first garment portion is engaged in a sound-reducing state. The garment also includes a second garment portion operable with the first garment portion, for example, where the first garment portion is a hood and the second garment portion is the body of a hooded sweatshirt. In the sound-reducing state, the sound-absorbing material reduces an amount of sound passing through the sound-absorbing material into the substantially enclosed reduced-sound zone.

### CROSS-REFERENCE TO RELATED APPLICATIONS

This application derives the benefit of the filing date of U.S. Provisional Patent Application No. 62/103,356, filed Jan. 14, 2015, the content of which provisional application is incorporated herein by reference.

### BACKGROUND OF THE INVENTION

The present invention is directed to a garment formed with a lining of material that serves as a sound absorbing layer.

Many garments are currently available with an added layer of material for a specific function, such as heat insulation or light reflection, when a basic garment without added features does not achieve desired technical or aesthetic goals. U.S. Pat. No. 8,625,812 (the '812 patent) discloses headwear that acoustically compensates or dampens detected acoustic signals in order to reduce the noise/sound that passes into a garment, such as a hooded sweatshirt. The acoustic management system of the '812 patent, however, is based on active noise cancellation and if the active noise cancellation is removed or underpowered, the hood would not and could not work as intended by the present invention.

U.S. Pat. No. 8,590,539 discloses a headgear-ear wear assembly, but does not refer to reduction of ambient or other sound proximate a wearer/user, nor under its present design would it be capable of doing so.

US Published Application Serial, No. 2015/0041243 discloses a hearing protection system, which may be attached to a cap and requires effort to put into a position that would protect the ears from noise. It is also not easily disguised as a typical wearable garment so would not be desirable for daily use in public environments. Most important is that the known hearing protection system does not allow for one to be aware of ongoing activity while also reducing noise and therefore dangerous to wear as a pedestrian or while operating a vehicle. The hearing protection system is limited in utility because it is cumbersome and offers no reduced sound zone about the user's ears.

U.S. Pat. No. 9,190,045 discloses a noise absorbent fabric for vehicles. Such fabric is generally non-woven and not used for garments. Even if sewed into a garment, it would not function to provide a reduced sound zone without the complete design of this present invention. This patent and many more of this category address a pressure wave by some form of an active or electric processing system, whereas the present invention is a clever passive system of noise reduction. U.S. Pat. No. 9,186,092 discloses a system garment and method that incorporate sensors used to measure or monitor pressure or forces in feet, the stump of limbs of an amputee, etc.

### SUMMARY OF THE INVENTION

The present invention overcomes the shortcomings of known arts, such as those mentioned above to provide a simple but comfortable everyday garment to passively reduce or enhance sound impinging on the wearer's ears.

To that end, the present invention provides a garment that is arranged with a garment portion configured to form a reduced-sound inner volume around a wearer's head, in particular, including the wearer's ears, when the inner volume is substantially closed, for example, such as by tying drawstrings tight to substantially close the inner volume. The garment wearer/user organically experiences a sound-reduced state. Depending on the configuration of the material from which the garment is formed (e.g., layered), or a design of a specific garment, the invention offers the wearer with varying levels of sound reduction in addition to aesthetic or additional functions. The garment includes a first garment portion, such as a hood portion of a sweatshirt and a second portion, such as the trunk portion of the sweatshirt, which second garment portion is operable with the first garment portion.

In an embodiment, the invention provides a garment engaged in a sound-reducing state. The garment includes a first garment portion formed from a material that includes a sound-absorbing material. The first garment portion is configured to surround part of a wearer's head and form a substantially enclosed reduced-sound zone about the user's ears when the first garment portion is engaged in a sound-reducing state. A second garment is portion operable with the first garment portion, for example, the first garment portion and the second garment portion detachably connect, such as in a hooded sweatshirt, where the hood is detachable. A zipper, buttons and Velcro embody devices for the detachable connection. In the sound-reducing state, the sound-absorbing material reduces an amount of sound passing through the sound-absorbing material into the substantially enclosed reduced-sound zone.

The material forming the first garment portion preferably comprises a base layer upon which the sound absorbing material is provided. Alternatively, the material forming the first garment portion comprises a base layer treated with the sound absorbing material or, the material forming the first garment portion comprises a base layer and wherein the sound absorbing material is impregnated into or otherwise integrated with the base layer. For that matter, the base layer may be formed as a natural or synthetic material.

In one form, the garment is a hooded sweatshirt, where the first garment portion is a detachable hood and the detachable hood is deployed to form the substantially enclosed reduced-sound zone. The reduced-sound zone minimizes an amount of sound entering the reduced-sound zone from outside the garment and reduces an amount of sound exiting the zone from inside the garment. In one form, the first garment portion and the second garment portion comprises the same material.

In another embodiment, the garment includes small cups in or proximate the reduced-sound zone, for receiving and directing sound towards the wearer's ears. The small cups are arranged to be tight to the wearer's ear at the back of the small cups. The small cups are open to maintaining a space between a face of the wearer the garment in a direction that the wearer is facing, for receiving and amplifying sound directed to a wearer. In an alternative embodiment, the small cups are arranged in or proximate the reduced-sound zone, in a way that inhibits sound going towards the wearer's ears. Preferably, the small cups are arranged to be tight to the wearer's ear all around the small cups. The small cups, or passive amplifiers, are secured to the hood by Velcro, an internal band, or any other method that the user finds as convenient.

In another form, the first garment portion is a scarf and the scarf is engaged to form the substantially enclosed reduce-sound zone. In a variation, the scarf is part of another system, such as a hat, where the scarf comprises the first garment portion, which defines the inner, sound-reduced volume and the hat comprises the second garment portion, the hat and scarf embodying the inventive garment. In another embodiment, the garment is the hooded sweatshirt where the first garment portion is the hood and the hood is substantially forward of the face creating a passive muffler. To that end, the hood is closed, creating the substantially forward of the face creating the passive muffler.

The sound absorbing lining, or sheet material formed with the sound absorbing layer may be any type of material that is flexible and can be made into a wearable garment, including weaves and knits, as well as organic or inorganic polymer sheets.

The sound absorbing material that faces the ear may be any type of material that absorbs or inhibits sound energy when sound waves pass by or through the material, as opposed to reflecting or channeling the sound energy toward the ear. For example, LUMITEX® sound absorbing material, manufactured by or for, distributed and/or sold by SoundSense LLC, Wainscott, N.Y., may he used as a sound absorbing material (e.g., a sound absorbing material layer) and/or as a sound barrier layer according to the invention. A two-layer system (of sheet material, for example) having a barrier layer and an absorbing layer, both with and without a base layer, is an inventive embodiment. For a garment, however, such a construction typically is heavier than two-layer solutions, so are preferred for limited applications.

The sound absorbing material is adhered to or otherwise arranged to be integral with at least a part of the material forming a garment, the part that is arranged or configured to be closed to form the sound-reduced inner volume. In one form, the material is formed from a base sheet material, such as a weave, where the sound absorbing material is arranged on the base sheet, in a portion of the garment that will be proximate the ear and the area between the ear and the entrance area(s) of the acoustic wave. The sound absorbing material can be attached by any standard means of garment assembly, for example, comprised of a single layer knit or woven cloth formed using natural or polymeric (synthetic) materials or any other natural or synthetic materials.

The resulting sheet material with the sound absorbing layer is preferably flexible so that it may be configured to function as a boundary to noise sound, separating an environment in which there is an undesirable sound level (such as an external environment) from an environment where a lower ambient sound level is desired (or required). Such an internal environment can be created by substantially enclosing the immediate space of an individual with the sheet material soundproofed with the sound absorbing layer.

Of course, the internal environment enclosed with the sheet material covered with the sound absorbing layer also operates to absorb sound generated in the internal environment so that noise level therein is lowered substantially. This will be useful in public instances such as a user listening to headphones at a loud volume or snoring.

When this same configuration is also intended to enhance the audibility of speech, cup shaped inserts, or passive amplifiers, are secured to the acoustic material. This configuration has an absorber reducing noise entering the garment, but amplifying the sound in the direction that the head faces. These cup shaped inserts surround the ear except for an opening directed to what is before the user's eyes/face, similar in concept to horse blinders. Sound directed to the user is readily received at the opening leading directly to the ear.

Another configuration these passive amplifiers are turned to enclose the ear from the front, top and bottom. In this configuration the ear is further protected from sounds entering the hooded sound reduced zone.

### BRIEF DESCRIPTION OF THE DRAWINGS

The invention will be described in conjunction with the following drawings in which like reference numerals designate like elements and wherein:

FIG. 1A Depicts a plan view of a surface of a sound attenuating sheet material 10 formed by adhering sound absorbing material 12 to approximately one half of one surface of the sheet of material 14;

FIG. 1B Shows a side cutaway view of the sheet 10 along the cut A-A′ depicted in FIG. 1A;

FIG. 2 Depicts a hooded sweatshirt (or hoodie) constructed with a sound attenuating cloth prepared to include sound absorbing material on one side (as described above), at least in a head portion;

FIG. 3 Shows a top garment portion of a hooded sweatshirt that includes small cups proximate a wearer's ears to receive and direct sound;

FIG. 4 Shows a hat with a garment section that is formed with a layer of sound absorbing material; and

FIG. 5 Depicts a material sheet formed to include sound absorbing material.

### DETAILED DESCRIPTION OF THE INVENTION

The following is a detailed description and drawings of example embodiments of the invention depicted. The example embodiments are presented in such detail as to clearly communicate the invention and are designed to make such embodiments obvious to a person of ordinary skill in the art. The amount of detail offered is not intended to limit the anticipated variations of embodiments, but to cover all modifications, equivalents, and alternatives falling within the spirit and scope of the present invention, as defined by the appended claims.

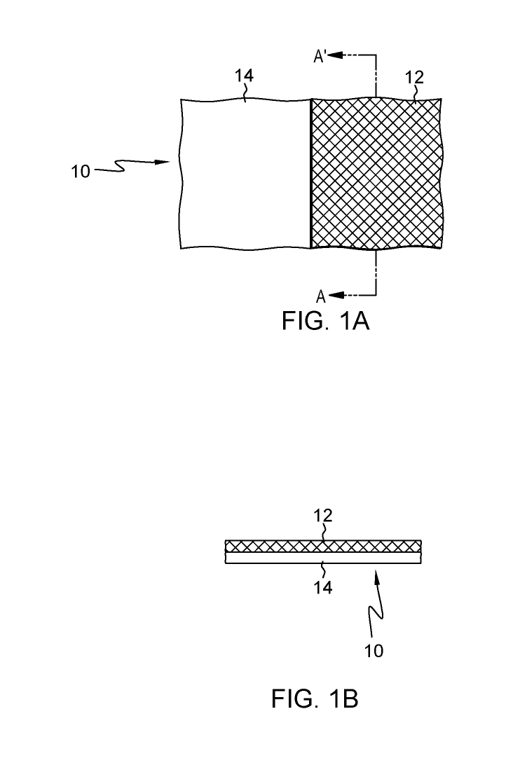

FIG. 1A depicts a plan view of a surface of a sound attenuating sheet material 10 formed with a layer of sound absorbing material 12 covering a base sheet material 14. The portion of the base sheet material 14 with the layer of sound absorbing material 12 is approximately one half the size of the entire surface of the sound attenuating sheet material, as shown, FIG. 1B shows a side cutaway view of the sound attenuating sheet material 10 along the cut A-A′ depicted in FIG. 1A.

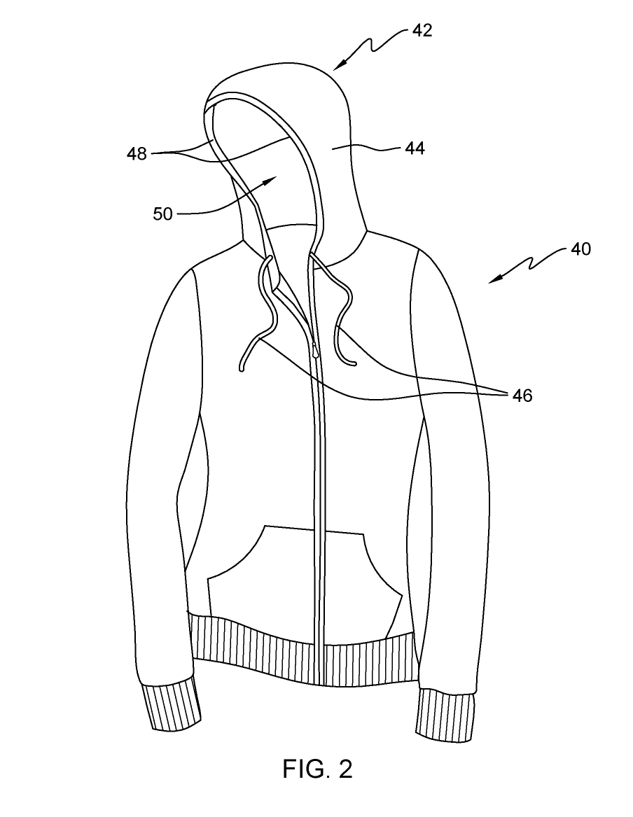

FIG. 2 depicts an exemplary garment in the form of a hooded sweatshirt (or hoodie) 40 constructed with a sound attenuating cloth prepared to include a layer of the sound absorbing material on one side of a base layer (as described above), at least in a head portion 42 of the sweatshirt 40. The sound absorbing material is on the inner surface of sound-attenuating cloth 44, from which the head portion is made. The head portion 44 may be integral with the remainder of the hooded sweatshirt, or may be detachable using a zipper, Velcro, etc., without limitation. For that matter, the sound attenuating cloth may comprise a hood portion 42, may be attached directly into the hood portion made of common material such as cotton, or may be a removable (separable, for example, using Velcro), both the lining and the hood itself,

During use, the user is to tighten strings 46, which are part of the head portion 42 formed with the sound attenuating cloth with the sound absorbing material on one side (the inner side as shown), pressing the edges of the head portion 48 against the user's face 50. These pull strings can be of any diameter or width and can also have a further closure attachment at the neck. By substantially closing the space using the strings, the sound-reduced inner volume is formed. Such movement forms a quasi-barrier to some sounds (in reliance on the sound absorbing quality of the material comprising the head portion) that might otherwise enter the space surrounded by the head portion 48 and proximate the ears and/or move through the sound attenuating material covering the ears.

The hooded sweatshirt 40 (with or without a zipper), as would any other garment constructed with the sound-attenuating cloth, has a look and feel like a normal sweatshirt, but the hidden design feature serves to reduce noise levels when in loud settings, such as public transportation or communal study environments.

An acoustic hood, hat or scarf designed to noticeably reduce surrounding noise has unlimited possibilities. Some examples are:
a. Hooded garments, such as a sweatshirt, could be worn on planes buses, public transportation or any public space to facilitate the wearer sleeping, reduce disturbance of a crying child or conversation occurring nearby, promote concentration while reading, or to enhance headphones audibility, etc.
b. Open work/office spaces where one is unable to control the volume of environmental noise that is considered to be a distraction and would inhibit productivity.
c. Educational environments where certain noises may be distracting and cause the reduction in retention of read material, or ability to focus on an exam, etc.
d. Autistic children or adults with noise sensitivity who rely on sensory control in unpredictable public environments, such as street, shopping malls, etc.
e. People of ill health, such as those with thyroid problems where noise sensitivity disturbs their sense of well-being.

Individuals that are regularly exposed to high noise levels due to occupation, such as the music industry or in a factory. Such vocations give little to no opportunity to avoid volumes or frequencies greater than recommended by OSHA. Often hearing protection is required but not always used as earplugs easy to misplace or are considered uncomfortable. NOTE: all garments in the noise reduction only category come with the criteria range from reducing the noise to inhibiting the noise.

The acoustic hood or hat with small cups to improve hearing clarity is an excellent support for the hearing impaired. This version can be used with, or independent of an acoustic hood for noise reduction with small speakers in the ear area, eliminating the need for earbuds or headphones. These speakers can be connected with a hardwire, or wirelessly. These speakers also can be incorporated into a hearing aid system.

An acoustic hood for noise reduction or hearing amplification can also provide radio frequency (RF) protection. In an embodiment, in combination with a layer of sound absorbing material provided on a base layer of a sheet material, and a layer or film of a material that attenuates RF energy, at least in some frequency ranges. Such garments may be helpful in minimizing exposure to RF and similar frequency wave energy, for example, in a region with multiple overhead power lines or radio towers.

While the embodiment was described with only a single noise or sound absorbing layer, the sheet material may also include multiple noise or sound absorbing layers on a base layer without deviating from the scope and spirit of the invention. The second noise absorbing layer can function as a decoupler for potential low frequency noises, or increase the NRC and/or the STC of the sound absorbing layer. The sound waves emitted from any source proximate the wearer (or a portion thereof) are absorbed by the noise absorbing layer before it enters any reduced sound compartment, like a small space for speaking on a cell phone enclosed within the acoustically treated portion of the hood (enclosure). With such a construction, a suitable sound transmission loss is achieved. The outer, inner or both noise absorbing layers preferably display a high NRC rated material, where “NRC” stands for noise reduction coefficient and represents the average amount of sound absorbed by the material. The NRC rating typically ranges from 0.01 to 1.0. NRC ratings above 1 (e.g., 1.03) are also possible. The higher the NRC rating of the material, the greater the sound absorption of the material. The noise or sound absorbing layer(s) is/are chosen based upon the characteristics of the particular mechanism and noise reduction goals.

Various materials are contemplated. In particular, the material forming the noise or sound absorbing layer(s) is/are chosen for sound absorbing qualities and ability to operate as a heat insulator to affect its properties as a garment, maintain heat. As such, the material forming the layer for noise attenuation can differ from that forming the base layer. The layer may also be formed from a closely woven textile-like material formed from any suitable material provided the material has adequate sound absorbing properties and withstands a predetermined temperature. A suitable adhesive can be used to secure the sound or noise absorbing layer to a base material layer. When multiple noise absorbing layers are provided, the layers can be formed by either the same material or a different material. Alternatively, the noise absorbing layer can be connected to a base or barrier layer when the garment is formed. The connection between layers can be achieved specified to the design or function of the garment, and is not limited to methods of bonding, layering or quilting.

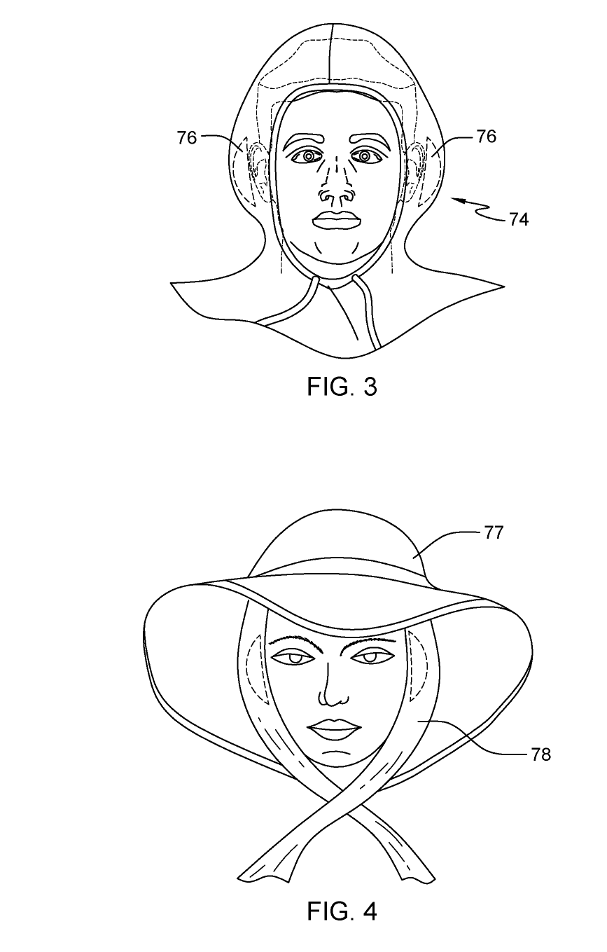

FIG. 3 shows a top garment portion 74 of a hooded sweatshirt (for example, the top or hooded part) that includes small cups 76 proximate a wearer's ears to receive and direct sound.

FIG. 4 shows a hat 77 with a garment section 78 that is formed with a layer of sound absorbing material.

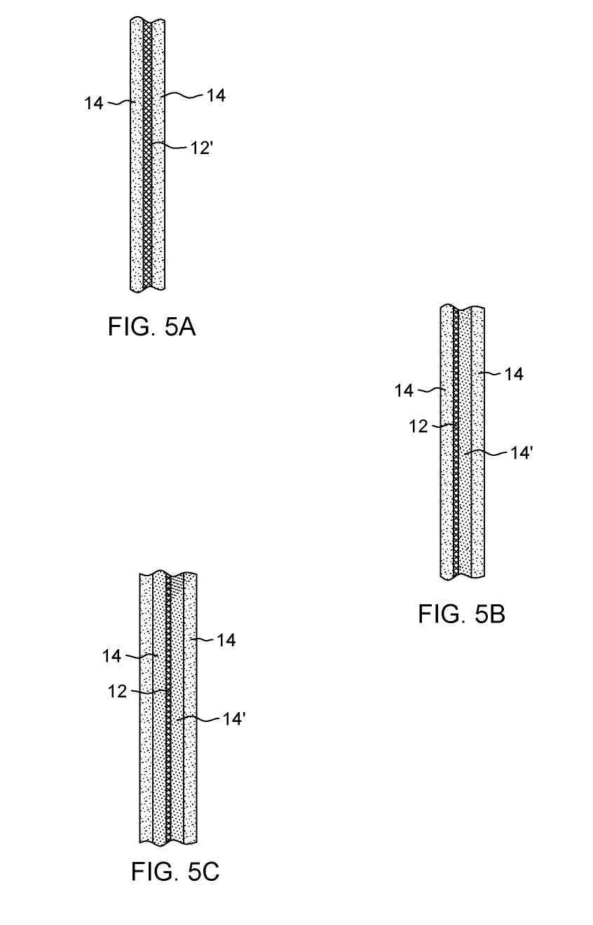

FIG. 5 shows a material sheet formed with any number of layers of materials including sound absorbing material. The layers may be said to be in sheets.

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

What is claimed is:

1. A garment engaged in a sound-reducing state, comprising:
a first garment portion formed from a material that includes a sound-absorbing material, wherein the first garment portion is configured to surround part of a wearer's head and form a substantially enclosed reduced-sound zone about the user's ears when the first garment portion is engaged in a sound-reducing state; and
a second garment portion operable with the first garment portion;
wherein in the sound-reducing state, the sound-absorbing material reduces an amount of sound passing through the sound-absorbing material into the substantially enclosed reduced-sound zone.

2. The garment of claim 1, wherein the material forming the first garment portion comprises a base layer upon which the sound absorbing material is provided.

3. The garment of claim 1, wherein the material forming the first garment portion comprises a base layer treated with the sound absorbing material is provided.

4. The garment of claim 1 wherein the material forming the first garment portion comprises a base layer and wherein the sound absorbing material is impregnated into or otherwise integrated with the base layer.

5. The garment of claim 2, wherein the base layer is a natural or synthetic material.

6. The garment of claim 2, wherein the first garment portion is detachably connected to the second garment portion.

7. The garment of claim 6, comprising a hooded sweatshirt.

8. The garment of claim 7, wherein the first garment portion is a detachable hood and wherein the detachable hood is deployed to form the substantially enclosed reduced-sound zone.

9. The garment of claim 9, wherein the first garment portion and the second garment portion are detachably connected using a zipper or Velcro.

10. The garment of claim 1, wherein the reduced-sound zone minimizes an amount of sound entering the reduced-sound zone from outside the garment and an amount of sound exiting the zone from inside the garment.

11. The garment of claim 1, wherein the first garment portion and the second garment portion comprise the same material.

12. The garment of claim 1, further including small cups positioned in or proximate the reduced-sound zone in an arrangement for receiving and directing sound towards the wearer's ears.

13. The garment of claim 12, wherein the small cups are arranged to be tight to the wearer's ear at the back of the small cups.

14. The garment of claim 13, wherein the small cups are open and maintain a space between a face of the wearer and a wall of the first garment portion, the space for receiving and/or amplifying sound directed to the wearer.

15. The garment of claim 1, further including small cups in or proximate the reduced-sound zone, for inhibiting sound going towards the wearer's ears.

16. The garment of claim 15, wherein the small cups are arranged to be tight to the wearer's ear all around the small cups.

17. The garment of claim 1, wherein the first garment portion is a scarf and wherein the hood is engaged to form the substantially enclosed reduce-sound zone.

18. The garment of claim 17, wherein the scarf is part of another system, such as a hat.

19. The garment of claim 1, wherein the hood is substantially forward of the face creating a passive muffler.

20. The garment of claim 19, wherein the hood is closed, creating the substantially forward of the face creating a passive muffler.
